(19) United States
(12) Patent Application Publication
(10) Pub. No.: US 2002/0106117 A1
(43) Pub. Date: Aug. 8, 2002

(54) Systems and computer software products for comparing microarray spot intensities

(76) Inventors: Daniel M. Bartell, San Carlos, CA (US); Wei-min Liu, Campbell, CA (US)

Correspondence Address: Chief IP Counsel - Legal Dept., Affymetrix, Inc., 3380 Central Expressway, Santa Clara, CA, 95051, US

(21) Appl. No.: 09/737,536

(22) Filed: Dec. 13, 2000

(51) Int. Cl. G06K 9/00; G06F 19/00; G01N 33/48; G01N 33/50; G06K 9/34

(52) U.S. Cl. 382/129; 702/19; 382/179

(57) ABSTRACT

Methods, systems and computer software products are provided for analyzing gene expression data using pixel intensities.

### FIELD OF INVENTION

This invention is related to bioinformatics and biological data analysis. Specifically, this invention provides methods, computer software products and systems for the analysis of biological data.

### BACKGROUND OF THE INVENTION

Many biological functions are carried out by regulating the expression levels of various genes, either through changes in the copy number of the genetic DNA, through changes in levels of transcription (e.g. through control of initiation, provision of RNA precursors, RNA processing, etc.) of particular genes, or through changes in protein synthesis. For example, control of the cell cycle and cell differentiation, as well as diseases, are characterized by the variations in the transcription levels of a group of genes.

Recently, massive parallel gene expression monitoring methods have been developed to monitor the expression of a large number of genes using nucleic acid array technology which was described in detail in, for example, U.S. Pat. No. 5,871,928; de Saizieu, et al., 1998, Bacteria Transcript Imaging by Hybridization of total RNA to Oligonucleotide Arrays, NATURE BIOTECHNOLOGY, 16:45-48; Wodicka et al., 1997, Genome-wide Expression Monitoring in Saccharomyces cerevisiae, NATURE BIOTECHNOLOGY 15:1359-1367; Lockhart et al., 1996, Expression Monitoring by Hybridization to High Density Oligonucleotide Arrays. NATURE BIOTECHNOLOGY 14:1675-1680; Lander, 1999, Array of Hope, NATURE-GENETICS, 21(suppl.), at 3.

Massive parallel gene expression monitoring experiments generate unprecedented amounts of information. For example, a commercially available GeneChip® array set is capable of monitoring the expression levels of approximately 6,500 murine genes and expressed sequence tags (ESTs) (Affymetrix, Inc, Santa Clara, Calif., USA). Array sets for approximately 60,000 human genes and EST clusters, 24,000 rat transcripts and EST clusters and arrays for other organisms are also available from Affymetrix. Effective analysis of the large amount of data may lead to the development of new drugs and new diagnostic tools. Therefore, there is a great demand in the art for methods for organizing, accessing and analyzing the vast amount of information collected using massive parallel gene expression monitoring methods.

### SUMMARY OF THE INVENTION

The current invention provides methods, systems and computer software products suitable for analyzing microarray spot data at the pixel level.

Microarrays may be made by, for example, robotically printing cDNA clone inserts onto a glass slide and subsequently hybridizing to two differentially fluorescently labeled samples. The samples may be a pools of cDNAs, which are generated after isolating mRNA from cells or tissues in two states that one wishes to compare.

In one aspect of the invention, methods are provided for comparing a first microarray spot with a second microarray spot. The methods may include steps of providing a first plurality of intensity values (Sᵢ^{A}) for the first micro array spot and a second plurality of intensity values (Sₖ^{B}) for the second microarray spot; calculating a p value using Wilcoxon's rank sum test, where the p value is for a null hypothesis that θ=0 and an alternative hypothesis that θ>0, where θ is a test statistic for intensity difference between the first plurality and the second plurality; and indicating that the first microarray spot is different from the second microarray spot if the p value is greater than a significance level. The test statistic may be is median (Sᵢ^{A})-median( Sₖ^{B}). The significance level can be, for example, 0.01, 0.05 or 0.10. The first microarray spot and second microarray spot may be nucleic acid spots among at least 10, 50, 100, 200, 400, 500, 750, 1,000, 5,000, 10,000, 20,000, 30,000 or more nucleic acid spots on a substrate. Exemplary nucleic acid spots include cDNA spots or oligonucleotide spots (either synthesized on the substrate or spotted). In some embodiments, the methods may include combining first plurality and second plurality of intensity values if the p-value is greater than a significance level, such as p>0.5.

In another aspect of the invention, computer software products are provided for comparing a first microarray spot with a second microarray spot. The products comprise computer program code for inputing a first plurality of intensity values (Sᵢ^{A}) for the first microarray spot and a second plurality of intensity values (Sₖ^{B}) for the second microarray spot; computer program code for calculating a p value using Wilcoxon's rank sum test, where the p value is for a null hypothesis that θ=0 and an alternative hypothesis that the θ>0, where the θ is a test statistic for intensity difference between the first plurality and the second plurality; computer program code for indicating that the first microarray spot is different from the second microarray spot if the p value is greater than a significance level; and a computer readable media for storing the computer program codes. The testing statistic is median (Sᵢ^{A})-median(Sₖ^{B}). The significance level may be, for example, 0.01, 0.05 or 0.10. In preferred embodiments, the computer software products may include computer program code for accepting user's input or selection of the significance level. The computer software products are particularly useful for analyzing spotted nucleic acid arrays such as those having at least 10, 50, 100, 200, 400, 500, 750, 1,000, 5,000, 10,000, 20,000, 30,000 or more nucleic acid spots on a substrate. The nucleic acid spots may be cDNA spots or oligonucleotide spots. The oligonucleotide spots may be spotted or synthesized on the substrate. The computer software products may also include computer program code for combining first plurality and second plurality of intensity values if the p-value is greater than a significance level.

In yet another aspect, systems for comparing two microarray spots are provided. The systems may include a processor; and a memory being coupled to the processor, the memory storing a plurality of machine instructions that cause the processor to perform a plurality of logical steps when implemented by the processor, the logical steps including: inputing a first plurality of intensity values (Sᵢ^{A}) for the first microarray spot and a second plurality of intensity values (Sₖ^{B}) for the second microarray spot; calculating a p value using Wilcoxon's rank sum test, where the p value is for a null hypothesis that θ=0 and an alternative hypothesis that the θ>0, where the θ is a test statistic for intensity difference between the first plurality and the second plurality; and indicating that the first microarray spot is different from the second microarray spot if the p value is greater than a significance level. The testing statistic may be median (Sᵢ^{A})-median(Sₖ^{B}). The significance level may be 0.05. In some preferred embodiments, the steps further include accepting user's input or selection of the significance level.

The systems are particularly useful for analyzing spotted nucleic acid arrays such as those having at least 10, 50, 100, 200, 400, 500, 750, 1,000, 5,000, 10,000, 20,000, 30,000 or more nucleic acid spots on a substrate. The nucleic acid spots may be cDNA spots or oligonucleotide spots. The oligonucleotide spots may be spotted or synthesized on the substrate. The computer software products may also include computer program code for combining first plurality and second plurality of intensity values if the p-value is greater than a significance level.

Methods, computer software products and systems are also provided for determining whether a transcript is present in a biological sample using nucleic acid probe arrays that have probes designed to be complementary to the transcript (perfect match probe, PM) and probes that are designed to contain mismatch against the transcript (mismatch probe, MM). The methods include providing a plurality of perfect match pixel intensity values (PMᵢⱼ) and mismatch pixel intensity values (MMᵢₖ) for the transcript, where the PMᵢⱼ is the pixel intensity value for perfect match probe i and pixel j and MMᵢₖ is the pixel intensity value for mismatch probe i and pixel k; calculating a p-value using one-sided Wilcoxon's rank sum test, wherein the p-value is for a null hypothesis that (median(PMᵢⱼ)-median(MMᵢₖ))=a threshold value and an alternative hypothesis that (median(PMᵢⱼ)-median(MMₗₖ))>the threshold value; and indicating whether the transcript is present based upon the resulting p-value. In some embodiments, the threshold value is zero. In some other preferred embodiments, the threshold value is calculated using:

τ=c \sqrt{median(PMᵢ)} or τ=c₁√mean(PMᵢ)

where c is a constant.

The presence, marginal present or absence (detected, marginally detected or undetected) of a transcript may be called based upon the p-value and significance levels. Significance levels, α₁ and α₂ may be set such that: 0<α₁<α₂<0.5. Note that for the one-side test, if null hypothesis is true, the most likely observed p-value is 0.5, which is equivalent to 1 for the two-sided test. Let p be the p-value of one sided rank sum test. In preferred embodiments, if p<α₁, a “detected” call can be made (i.e., the expression of the target gene is detected in the sample). If α₁≦p<α₂, a marginally detected call may be made. If p≧α₂, “undetected call” may be made. The proper choice of significance levels and the thresholds can reduce false calls.

Some preferred embodiments of the computer software product for determining whether a transcript is present in a biological sample include computer program code for inputting a plurality of perfect match pixel intensity values (PMᵢⱼ) and mismatch pixel intensity values (MMᵢₖ) for the transcript, wherein the PMᵢⱼ is the pixel intensity value for perfect match probe i and pixel j and MMᵢₖ is the pixel intensity value for mismatch probe i and pixel k; computer software code for calculating a p-value using one-sided Wilcoxon's rank sum test, wherein the p-value is for a null hypothesis that (median(PMᵢⱼ)-median(MMᵢₖ))=a threshold value and an alternative hypothesis that (median(PMᵢⱼ)-median(MMᵢₖ))>threshold value; computer software code for indicating whether the transcript is present based upon said p-value; and a computer readable media for storing the codes.

In some embodiments, the threshold value is zero. In some other preferred embodiments, the threshold value is calculated using:

τ=c\sqrt{median(PMᵢ)} or τ=c₁\sqrt{mean(PMᵢ)}

where c is a constant.

The computer software product may also include code for indicating the presence, marginal presence or absence of the transcript based up the p-value and significance level. Appropriate significance level may be pre-set or inputted by a user.

Systems for comparing intensities for nucleic acid probes are also provided. The systems may include a processor; and a memory being coupled to the processor, the memory storing a plurality machine instructions that cause the processor to perform a plurality of logical steps when implemented by the processor, the logical steps including: providing a plurality of perfect match pixel intensity values (PMᵢⱼ) and mismatch pixel intensity values (MMᵢₖ) for the transcript, where PMᵢⱼ is the pixel intensity value for perfect match probe i and pixel j and MMᵢₖ is the pixel intensity value for mismatch probe i and pixel k;calculating a p-value using one-sided Wilcoxon's rank sum test, wherein the p-value is for a null hypothesis that (median(PMᵢⱼ)-median(MMᵢₖ))=a threshold value and an alternative hypothesis that said (median(PMᵢⱼ)-median(MMᵢₖ))>said threshold value; and indicating whether said transcript is present based upon said p-value.

In some embodiments, the threshold value is zero. In some other preferred embodiments, the threshold value is calculated using:

τ=c\sqrt{median(PMᵢ)} or τ=c₁\sqrt{mean(PMᵢ)}

where c is a constant.

The presence, marginal present or absence (detected, marginally detected or undetected) of a transcript may be called based upon the p-value and significance levels. Significance levels, α₁ and α₂ may be set such that: 0<α₁<α₂<0.5. Note that for the one-sided test, if null hypothesis is true, the most likely observed p-value is 0.5, which is equivalent to 1 for the two-sided test. Let p be the p-value of one sided rank sum test. In preferred embodiments, if p<α₁, a “detected” call can be made (i.e., the expression of the target gene is detected in the sample). If α₁≦p<α₂, a marginally detected call may be made. If P≧α₂, “undetected call” may be made. The proper choice of significance levels and the thresholds can reduce false calls.

### BRIEF DESCRIPTION OF THE DRAWINGS

The accompanying drawings, which are incorporated in and form a part of this specification, illustrate embodiments of the invention and, together with the description, serve to explain the principles of the invention:

FIG. 1 illustrates an example of a computer system that may be utilized to execute the software of an embodiment of the invention.

FIG. 2 illustrates a system block diagram of the computer system of FIG. 1.

FIG. 3 shows two microarray images.

FIG. 4 shows microarray spots.

### DETAILED DESCRIPTION OF THE PREFERRED EMBODIMENTS

Reference will now be made in detail to the preferred embodiments of the invention. While the invention will be described in conjunction with the preferred embodiments, it will be understood that they are not intended to limit the invention to these embodiments. On the contrary, the invention is intended to cover alternatives, modifications and equivalents, which may be included within the spirit and scope of the invention. All cited references, including patent and non-patent literature, are incorporated herein by reference in their entireties for all purposes.

I. Gene Expression Monitoring With High Density Oligonucleotide Probe Arrays

High density nucleic acid probe arrays, also referred to as “DNA Microarrays,” have become a method of choice for monitoring the expression of a large number of genes. As used herein, “Nucleic acids” may include any polymer or oligomer of nucleosides or nucleotides (polynucleotides or oligonucleotidies), which include pyrimidine and purine bases, preferably cytosine, thymine, and uracil, and adenine and guanine, respectively. See Albert L. Lehninger, PRINCIPLES OF BIOCHEMISTRY, at 793-800 (Worth Pub. 1982) and L. Stryer BIOCHEMISTRY, 4^{th} Ed., (March 1995), both incorporated by reference. “Nucleic acids” may include any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally-occurring sources or may be artificially or synthetically produced. In addition, the nucleic acids may be DNA or RNA, or a mixture thereof, and may exist permanently or transitionally in single-stranded or double-stranded form, including homoduplex, heteroduplex, and hybrid states.

“A target molecule” refers to a biological molecule of interest. The biological molecule of interest can be a ligand, receptor, peptide, nucleic acid (oligonucleotide or polynucleotide of RNA or DNA), or any other of the biological molecules listed in U.S. Pat. No. 5,445,934 at col. 5, line 66 to col. 7, line 51. For example, if transcripts of genes are the interest of an experiment, the target molecules would be the transcripts. Other examples include protein fragments, small molecules, etc. “Target nucleic acid” refers to a nucleic acid (often derived from a biological sample) of interest. Frequently, a target molecule is detected using one or more probes. As used herein, a “probe” is a molecule for detecting a target molecule. It can be any of the molecules in the same classes as the target referred to above. A probe may refer to a nucleic acid, such as an oligonucleotide, capable of binding to a target nucleic acid of complementary sequence through one or more types of chemical bonds, usually through complementary base pairing, usually through hydrogen bond formation. As used herein, a probe may include natural (i.e. A, G, U, C, or T) or modified bases (7-deazaguanosine, inosine, etc.). In addition, the bases in probes may be joined by a linkage other than a phosphodiester bond, so long as the bond does not interfere with hybridization. Thus, probes may be peptide nucleic acids in which the constituent bases are joined by peptide bonds rather than phosphodiester linkages. Other examples of probes include antibodies used to detect peptides or other molecules, any ligands for detecting its binding partners. When referring to target or probes as nucleic acids, it should be understood that these are illustrative embodiments that are not to limit the invention in any way.

In preferred embodiments, probes may be immobilized on substrates to create an array. An “array” may comprise a solid support with peptide or nucleic acid or other molecular probes attached to the support. Arrays typically comprise a plurality of different nucleic acids or peptide probes that are coupled to a surface of a substrate in different, known locations. These arrays, also described as “microarrays” or colloquially “chips” have been generally described in the art, for example, in Fodor et al., Science, 251:767-777 (1991), which is incorporated by reference for all purposes. Methods of forming high density arrays of oligonucleotides, peptides and other polymer sequences with a minimal number of synthetic steps are disclosed in, for example, U.S. Pat. Nos. 5,143,854, 5,252,743, 5,384,261, 5,405,783, 5,424,186, 5,429,807, 5,445,943, 5,510,270, 5,677,195, 5,571,639, 6,040,138, all incorporated herein by reference for all purposes. The oligonucleotide analogue array can be synthesized on a solid substrate by a variety of methods, including, but not limited to, light-directed chemical coupling, and mechanically directed coupling. See Pirrung et al., U.S. Pat. No. 5,143,854 (see also PCT Application No. WO 90/15070) and Fodor et al., PCT Publication Nos. WO 92/10092 and WO 93/09668, U.S. Pat. Nos. 5,677,195, 5,800,992 and 6,156,501 which disclose methods of forming vast arrays of peptides, oligonucleotides and other molecules using, for example, light-directed synthesis techniques. See also, Fodor et al., Science, 251, 767-77 (1991). These procedures for synthesis of polymer arrays are now referred to as VLSIPS™ procedures. Using the VLSIPS™ approach, one heterogeneous array of polymers is converted, through simultaneous coupling at a number of reaction sites, into a different heterogeneous array. See, U.S. Pat. Nos. 5,384,261 and 5,677,195.

Methods for making and using molecular probe arrays, particularly nucleic acid probe arrays are also disclosed in, for example, U.S. Pat. Nos. 5,143,854, 5,242,974, 5,252,743, 5,324,633, 5,384,261, 5,405,783, 5,409,810, 5,412,087, 5,424,186, 5,429,807, 5,445,934, 5,451,683, 5,482,867, 5,489,678, 5,491,074, 5,510,270, 5,527,681, 5,527,681, 5,541,061, 5,550,215, 5,554,501, 5,556,752, 5,556,961, 5,571,639, 5,583,211, 5,593,839, 5,599,695, 5,607,832, 5,624,711, 5,677,195, 5,744,101, 5,744,305, 5,753,788, 5,770,456, 5,770,722, 5,831,070, 5,856,101, 5,885,837, 5,889,165, 5,919,523, 5,922,591, 5,925,517, 5,658,734, 6,022,963, 6,150,147, 6,147,205, 6,153,743, 6,140,044 and D430024, all of which are incorporated by reference in their entireties for all purposes. Typically, a nucleic acid sample is a labeled with a signal moiety, such as a fluorescent label. The sample is hybridized with the array under appropriate conditions. The arrays are washed or otherwise processed to remove non-hybridized sample nucleic acids. The hybridization is then evaluated by detecting the distribution of the label on the chip. The distribution of label may be detected by scanning the arrays to determine fluorescence intensity distribution. Typically, the hybridization of each probe is reflected by several pixel intensities. The raw intensity data may be stored in a gray scale pixel intensity file. The GATC™ Consortium has specified several file formats for storing array intensity data. The final software specification is available at www.gatcconsortium.org and is incorporated herein by reference in its entirety. The pixel intensity files are usually large. For example, a GATC™ compatible image file may be approximately 50 Mb if there are about 5000 pixels on each of the horizontal and vertical axes and if a two byte integer is used for every pixel intensity. The pixels may be grouped into cells (see, GATC™ software specification). The probes in a cell are designed to have the same sequence (i.e., each cell is a probe area). A CEL file contains the statistics of a cell, e.g., the 75th percentile and standard deviation of intensities of pixels in a cell. The 75th percentile of pixel intensity of a cell is often used as the intensity of the cell. Methods for signal detection and processing of intensity data are additionally disclosed in, for example, U.S. Pat. Nos. 5,547,839, 5,578,832, 5,631,734, 5,800,992, 5,856,092, 5,936,324, 5,981,956, 6,025,601, 6,090,555, 6,141,096, 6,141,096, and 5,902,723. Methods for array based assays, computer software for data analysis and applications are additionally disclosed in, e.g., U.S. Pat. Nos. 5,527,670, 5,527,676, 5,545,531, 5,622,829, 5,631,128, 5,639,423, 5,646,039, 5,650,268, 5,654,155, 5,674,742, 5,710,000, 5,733,729, 5,795,716, 5,814,450, 5,821,328, 5,824,477, 5,834,252, 5,834,758, 5,837,832, 5,843,655, 5,856,086, 5,856,104, 5,856,174, 5,858,659, 5,861,242, 5,869,244, 5,871,928, 5,874,219, 5,902,723, 5,925,525, 5,928,905, 5,935,793, 5,945,334, 5,959,098, 5,968,730, 5,968,740, 5,974,164, 5,981,174, 5,981,185, 5,985,651, 6,013,440, 6,013,449, 6,020,135, 6,027,880, 6,027,894, 6,033,850, 6,033,860, 6,037,124, 6,040,138, 6,040,193, 6,043,080, 6,045,996, 6,050,719, 6,066,454, 6,083,697, 6,114,116, 6,114,122, 6,121,048, 6,124,102, 6,130,046, 6,132,580, 6,132,996 and 6,136,269, all of which are incorporated by reference in their entireties for all purposes.

Nucleic acid probe array technology, use of such arrays, analysis array based experiments, associated computer software, composition for making the array and practical applications of the nucleic acid arrays are also disclosed, for example, in the following U.S. patent applications Ser. Nos.: 07/838,607, 07/883,327, 07/978,940, 08/030,138, 08/082,937, 08/143,312, 081327,522, 081376,963, 08/440,742, 08/533,582, 08/643,822, 08/772,376, 09/013,596, 09/016,564, 09/019,882, 09/020,743, 09/030,028, 09/045,547, 09/060,922, 09/063,311, 09/076,575, 09/079,324, 09/086,285, 09/093,947, 09/097,675, 09/102,167, 09/102,986, 09/122,167, 09/122,169, 09/122,216, 09/122,304, 09/122,434, 09/126,645, 09/127,115, 09/132,368, 09/134,758, 09/138,958, 09/146,969, 09/148,210, 09/148,813, 09/170,847, 09/172,190, 09/174,364, 09/199,655, 09/203,677, 09/256,301, 09/285,658, 09/294,293, 09/318,775, 09/326,137, 09/326,374, 091341,302, 09/354,935, 09/358,664, 09/373,984, 09/377,907, 09/383,986, 09/394,230, 09/396,196, 09/418,044, 09/418,946, 09/420,805, 09/428,350, 09/431,964,09/445,734, 09/464,350, 09/475,209, 09/502,048, 09/510,643, 09/513,300, 09/516,388, 09/528,414, 09/535,142, 09/544,627, 09/620,780,09/640,962, 09/641,081, 09/670,510, 09/685,011, and 09/693,204 and in the following Patent Cooperative Treaty (PCT) applications/publications: PCT/NL90/00081, PCT/GB91/00066, PCT/US91/08693, PCT/US91/09226, PCT/US91/09217, WO/93/10161, PCT/US92/10183, PCT/GB93/00147, PCT/US93/01152, WO/93/22680, PCT/US93/04145, PCT/US93/08015, PCT/US94/07106, PCT/US94/12305, PCT/GB95/00542, PCT/US95/07377, PCT/US95/02024, PCT/US96/05480, PCT/US96/11147, PCT/US96/14839, PCT/US96/15606, PCT/US97/01603, PCT/US97/02102, PCT/GB97/005566, PCT/US97/06535, PCT/GB97/01148, PCT/GB97/01258, PCT/US97/08319, PCT/US97/08446, PCT/US97/10365, PCT/US97/17002, PCT/US97/16738, PCT/US97/19665, PCT/US97/20313, PCT/US97/21209, PCT/US97/21782, PCT/US97/23360, PCT/US98/06414, PCT/US98/01206, PCT/GB98/00975, PCT/US98/04280, PCT/US98/04571, PCT/US98/05438, PCT/US98/05451, PCT/US98/12442, PCT/US98/12779, PCT/US98/12930, PCT/US98/13949, PCT/US98/15151, PCT/US98/15469, PCT/US98/15458, PCT/US98/15456, PCT/US98/16971, PCT/US98/16686, PCT/US99/19069, PCT/US98/18873, PCT/US98/18541, PCT/US98/19325, PCT/US98/22966, PCT/US98/26925, PCT/US98/27405 and PCT/IB99/00048, all of which are incorporated by reference in their entireties for all purposes. All the above cited patent applications and other references cited throughout this specification are incorporated herein by reference in their entireties for all purposes.

The embodiments of the invention will be described using GeneChip® high oligonucleotide density probe arrays (available from Affymetrix, Inc., Santa Clara, Calif. USA) as exemplary embodiments. One of skill in the art would appreciate that the embodiments of the invention are not limited to high density oligonucleotide probe arrays. In contrast, the embodiments of the invention are useful for analyzing any parallel large scale biological analysis, such as those using nucleic acid probe array, protein arrays, etc.

Gene expression monitoring using GeneChip® high density oligonucleotide probe arrays are described in, for example, Lockhart et al., 1996, Expression Monitoring By Hybridization to High Density Oligonucleotide Arrays, Nature Biotechnology 14:1675-1680; U.S. Pat. Nos. 6,040,138 and 5,800,992, all incorporated herein by reference in their entireties for all purposes.

In the preferred embodiment, oligonucleotide probes are synthesized directly on the surface of the array using photolithography and combinatorial chemistry as disclosed in several patents previous incorporated by reference. In such embodiments, a single rectangular-shaped feature on an array contains one type of probe. Probes are selected to be specific for a desired target. Methods for selecting probe sequences are disclosed in, for example, U.S. patent application Ser. Nos.______, Attorney Docket Number 3359; ______, filed Nov. 21, 2000, Attorney Docket Number 3367, filed Nov. 21, 2000, and______, Attorney Docket Number 3373, filed Nov. 21, 2000, all incorporated herein by reference in their entireties for all purposes.

In a preferred embodiment, oligonucleotide probes in the high density array are selected to bind specifically to the nucleic acid target to which they are directed with miminimal non-specific binding or cross-hybridization under the particular hybridization conditions utilized. Because the high density arrays of this invention can contain in excess of 1,000,000 different probes, it is possible to provide every probe of a characteristic length that binds to a particular nucleic acid sequence. Thus, for example, the high density array can contain every possible 20 mer sequence complementary to an IL-2 mRNA. There, however, may exist 20 mer subsequences that are not unique to the IL-2 mRNA. Probes directed to these subsequences are expected to cross hybridize with occurrences of their complementary sequence in other regions of the sample genome. Similarly, other probes simply may not hybridize effectively under the hybridization conditions (e.g., due to secondary structure, or interactions with the substrate or other probes). Thus, in a preferred embodiment, the probes that show such poor specificity or hybridization efficiency are identified and may not be included either in the high density array itself (e.g., during fabrication of the array) or in the post-hybridization data analysis.

Probes as short as 15, 20, 25 or 30 nucleotides are sufficient to hybridize to a subsequence of a gene and that, for most genes, there is a set of probes that performs well across a wide range of target nucleic acid concentrations. In a preferred embodiment, it is desirable to choose a preferred or “optimum” subset of probes for each gene before synthesizing the high density array.

In some preferred embodiments, the expression of a particular transcript may be detected by a plurality of probes, typically, up to 5, 10, 15, 20, 30 or 40 probes. Each of the probes may be designed to detect different sub-regions of the transcript. However, probes may overlap over targeted regions.

In some preferred embodiments, each target sub-region is detected using two probes: a perfect match (PM) probe that is designed to be completely complementary to a reference or target sequence. In some other embodiments, a PM probe may be substantially complementary to the reference sequence. A mismatch (MM) probe is a probe that is designed to be complementary to a reference sequence except for some mismatches that may significantly affect the hybridization between the probe and its target sequence. In preferred embodiments, MM probes are designed to be complementary to a reference sequence except for a homomeric base mismatch at the central (e.g., 13^{th} in a 25 base probe) position. Mismatch probes are normally used as controls for cross-hybridization. A probe pair is usually composed of a PM and its corresponding MM probe. The difference between PM and MM provides an intensity difference in a probe pair.

In some other applications, spotted DNA microarrays may be used to comparatively analyze patterns of mRNA expression. Se U.S. Pat. No. 6,040,193. Microarrays may be made by, for example, robotically printing cDNA clone inserts onto a glass slide and subsequently hybridizing to two differently fluorescently labeled samples. See U.S. Pat. No. 5,599,695. The samples may be pools of cDNAs, which are generated after isolating mRNA from cells or tissues in two states that one wishes to compare. Resulting fluorescent intensities may be produced using a laser confocal fluorescent microscope, and intensity ratios between two colors are obtained following image processing. For an extensive review of the microarray technology, see Mark Schena, 2000, Microarray Biochip Technology, Eaton Publishing, ISBN 1-881299-37-6), which is incorporated herewith by reference in its entirety for all purposes.

II. Data Analysis Systems

In one aspect of the invention, methods, computer software products and systems are provided for computational analysis of microarray intensity data for determining the presence or absence of genes in a given biological sample. Accordingly, the present invention may take the form of data analysis systems, methods, analysis software, etc. Software written according to the present invention is to be stored in some form of computer readable medium, such as memory, or CD-ROM, or transmitted over a network, and executed by a processor. For a description of basic computer systems and computer networks, see, e.g., Introduction to Computing Systems: From Bits and Gates to C and Beyond by Yale N. Patt, Sanjay J. Patel, 1st edition (Jan. 15, 2000) McGraw Hill Text; ISBN: 0072376902; and Introduction to Client/Server Systems : A Practical Guide for Systems Professionals by Paul E. Renaud, 2nd edition (June 1996), John Wiley & Sons; ISBN: 0471133337.

Computer software products may be written in any of various suitable programming languages, such as C, C++, C# (Microsoft®), Fortran, Perl, MatLab (MathWorks, www.mathworks.com), SAS, SPSS and Java. The computer software product may be an independent application with data input and data display modules. Alternatively, the computer software products may be classes that may be instantiated as distributed objects. The computer software products may also be component software such as Java Beans (Sun Microsystem), Enterprise Java Beans (EJB, Sun Microsystems), or Microsoft® COM/DCOM (Microsoft®), etc.

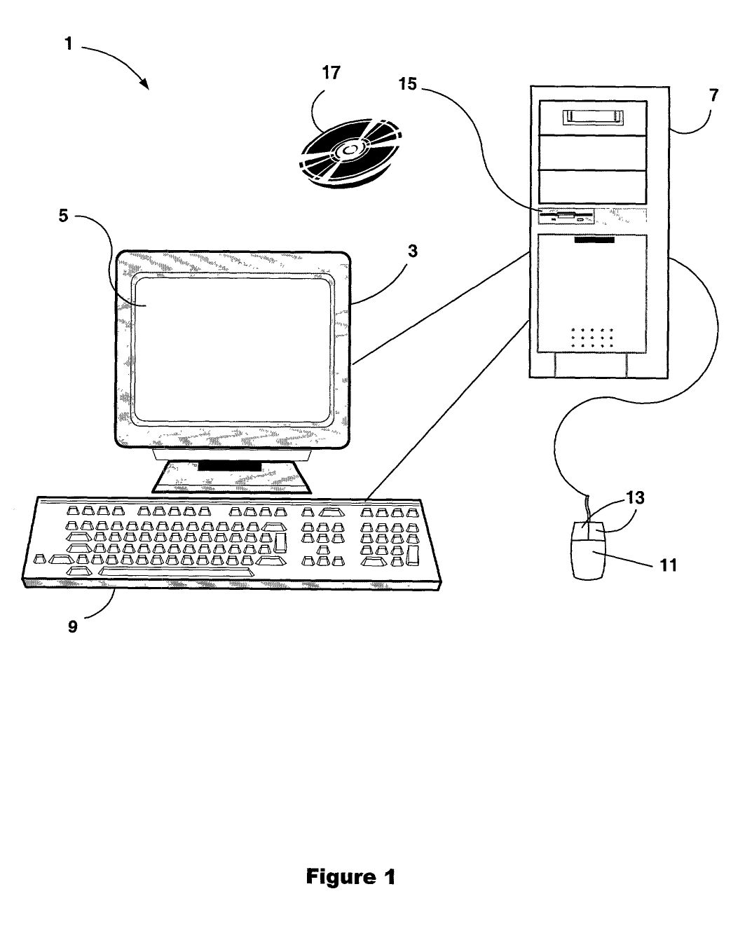

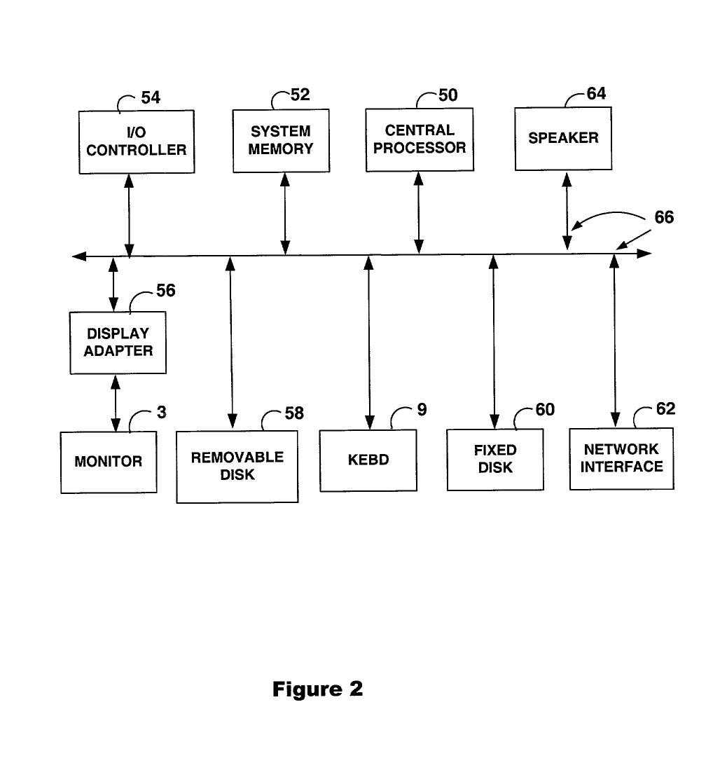

FIG. 1 illustrates an example of a computer system that may be used to execute the software of an embodiment of the invention. FIG. 1 shows a computer system 1 that includes a display 3, screen 5, cabinet 7, keyboard 9, and mouse 11. Mouse 11 may have one or more buttons for interacting with a graphic user interface. Cabinet 7 houses a CD-ROM or DVD-ROM drive 13, system memory and a hard drive (see FIG. 2) which may be utilized to store and retrieve software programs incorporating computer code that implements the invention, data for use with the invention and the like. Although a CD 17 is shown as an exemplary computer readable medium, other computer readable storage media including floppy disk, tape, flash memory, system memory, and hard drive may be utilized. Additionally, a data signal embodied in a carrier wave (e.g., in a network including the Internet) may be the computer readable storage medium.

FIG. 2 shows a system block diagram of computer system 1 used to execute the software of an embodiment of the invention. As in FIG. 1, computer system 1 includes monitor 3, keyboard 9, and mouse 11. Computer system 1 further includes subsystems such as a central processor 50, system memory 52, fixed storage 60 (e.g., hard drive), removable storage 58 (e.g., CD-ROM), display adapter 56, speakers 64, and network interface 62. Other computer systems suitable for use with the invention may include additional or fewer subsystems. For example, another computer system may include more than one processor 50 or a cache memory. Computer systems suitable for use with the invention may also be embedded in a measurement instrument.

III. Pixel Intensity Comparison

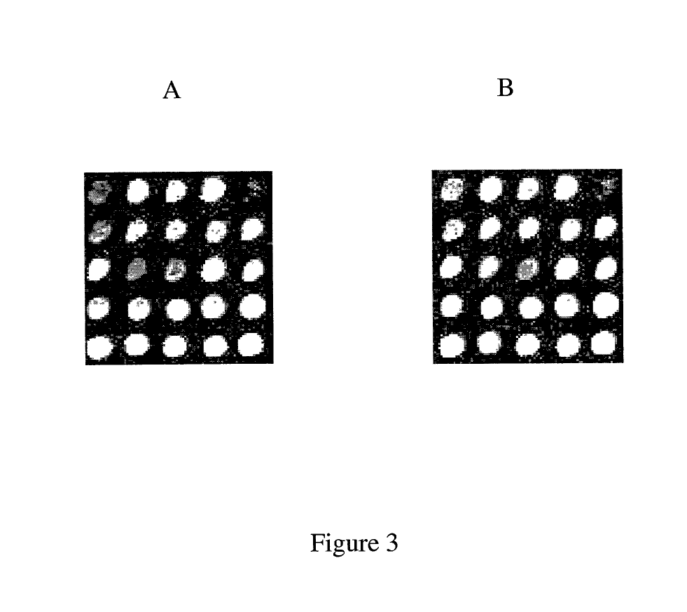

Computational analysis of microarray spot intensity data to extract probe intensities at each cDNA target location is an important part of the microarray data analysis and provides a foundation for further high-level analysis. One important question in such analysis is whether the spots have different intensities. FIGS. 3A and 3B show examplary microarray image data. Each spot of the image represents a cDNA probe immobilized on a substrate. Comparing between images in FIGS. 3A and 3B, the upper left spots are clearly of different intensities. However, the center spots appear similar in intensity and additional analysis is needed to determine whether they have different intensities.

In one aspect of the invention, methods, computer software and systems are provided to determine the probability that the microarray spots have different intensities. The methods include steps for computing p-values using non-parametric statistics, particularly Wilconxon's Rank Sum Test.

Nonparametric statistical methods are powerful tools for computing exact p-values when the distribution of original data is unknown (e.g., Hogg R V, Tanis E A (1997) Probability and Statistical Inference (fifth edition), Upper Saddle River, N.J.:Prentice-Hall, Inc.; Hollander M, Wolfe D A (1999). Nonparametric Statistical Methods (second edition), New York: John Wiley & Sons, Inc., both incorporated herein by reference for all purposes).

Many nonparametric methods use ranks or signs of data, and hence are insensitive to outliers. Their assumptions about the distributions of the original data are much weaker than those of parametric methods. Therefore, they can be applied to more general situations. Nonparametric statistics has been used to determine whether a gene is expressed in a sample, see, e.g., Provisional Application Ser. No., 60/189,558, filed on Mar. 15, 2000 and U.S. patent application Ser. No.______, Attorney Docket Number 3298.1, filed Dec. 12, 2000, both incorporated herein by reference in their entireties for all purposes.

Wilcoxon's rank sum test can be applied to analyze two data sets of different size, such as intensity data from spotted arrays. In such arrays, the size of spots (usually, each spot represents one probe), and thus the number of pixels, typically varies. In addition, the pixel intensities in a pair of spots are not paired. Therefore, Wilcoxon's test for two samples or Wilcoxon's rank sum test may be appropriate (e.g., Hogg R V, Tanis E A (1997) Probability and Statistical Inference (fifth edition), Upper Saddle River, N.J.:Prentice-Hall, Inc.; Hollander M, Wolfe D A (1999). Nonparametric Statistical Methods (second edition), New York: John Wiley & Sons, Inc.; Wilconxon et al., 1973, Critical Values and probability levels for the Wilcoxon Rank Sum Test and the Wilcoxon Signed Ranks Test. In Selected Tables in Mathematical Statistics, Volume 1, Edited Harter and Owen, Providence, R.I. American Mathematical Society and Institute of Mathematical Statistics, Wilcoxon, F. Individual Comparisons by Ranking Methods, Biometrics 1:80-83 (1945); Mann and Whitney, On a test of whether one or two random variables is stochastically larger than the other. Ann. Math. Stat. 18:50-60 (1947), all incorporated herewith by reference in their entireties for all purposes).

In some embodiments, the pixel intensities for the two sets of pixel intensity data are organized as follows. Assign all the intensities from one of the spots to set Sᵢ^{A}. Assign all intensities from the other spot to Sₖ^{B}. n is the size of Sᵢ^{A}. m is the size of Sₖ^{B}. Let the i-th pixel intensity in the first spot be Sᵢ^{A} (i=1, . . . n). Let the k-th pixel intensity in the second spot be S B (k=1, . . . m).

The combined pixel intensity data, Sᵢ^{A} and Sₖ^{B} can be sorted and ranked with integers 1,2, . . . p, where total number of pixels in the first and second spots is p=m+n. If there are ties, the average of the integer ranks for all elements in a tie group may be used. Let the rank of Sᵢ^{A} be Rᵢ^{A} and the rank of Sᵢ^{B} be Rᵢ^{B}. The rank sum may calculated as

$$W = {\sum }_{j = 1}^{n} {R}_{j}^{A} \qquad ( 1 )$$

The exact p-values of the observed W can be calculated. When the number of pixels, n and m, in the two spots are large, the asymptotic normal approximation may be used.

The Wilconxon's rank sum test may also be used to analyze oligonucleotide probe arrays. In some embodiments, pixel intensities in a pair of cells, the data are not really paired. Therefore, Wilcoxon's test for two samples may be used. In some embodiments, Wilcoxon's rank sum test is used to analyze paired PM and MM probes. In a block of n probe pairs (also known as atoms) for detecting a gene (typically 10, 15, or 20 probe pairs). Each probe pair typically consists of two cells, one has the sequence designed to be perfectly matching the target sequence and the other has the sequence designed to be mismatching the target sequence, preferably at only a single nucleotide location (usually at the center of the sequence segment).

Let PMᵢⱼ be the intensity of pixel j in the perfect match cell of atom i (j=1, . . . pᵢ) where pᵢ is the number of pixels used in this cell. Similarly, let MMᵢₖ be the intensity of pixel k in the mismatch cell of atom i (k=1, . . . ,mᵢ), where mᵢ is the number of pixels used in the cell. Note that the number of pixels pᵢ and mᵢ do not have to be the same. The combined intensity data PMᵢⱼ and MMᵢⱼ may be sorted and ranked with integers 1,2, . . . , Nᵢ, where Nᵢ=pᵢ+mᵢ is the total number of pixels used in these two cells. If there are ties, the average of integer ranks for all elements in a tie group may be used. Let the rank of PMᵢⱼ be rᵢⱼ^{(P)} and the rank of MMᵢₖ be rᵢₖ^{(m)}. Calculate Wilcoxon's rank sum

$${W}_{2} ( i ) = {\sum }_{j = 1}^{pi} {r}_{ij}^{( p )} \qquad ( 2 )$$

The exact p-values of observed W₂(i) can be calculated. When the number of pixels, pᵢ and mᵢ, in the two cells are large, the asymptotic normal approximation may be used. Since W₂(i) has the mean and variance

$$\begin{matrix}{μ}_{w2 ( i )} = \frac{{p}_{i} ( {N}_{i} + 1 )}{2} , & ( 3 ) \\ {V}_{w2 ( t )} = \frac{{p}_{i} {m}_{i}}{12 {N}_{i} ( {N}_{i} - 1 )} [ {N}_{i} ( {N}_{i}^{2} - 1 ) - {\sum }_{k = 1}^{gi} {t}_{ik} ( {t}_{ik}^{2} - 1 ) ] , & ( 4 )\end{matrix}$$

where gi is the number of tied groups of the i-th atom, and tᵢₖ is the number of tied entries in the k-th tied group of the i-th atom. Then the statistic

$${W}_{2}^{*} ( i ) = \frac{{W}_{2} ( i ) - μ {W}_{2} ( i )}{\sqrt{{V}_{{W}_{2 ( i )}}}} \qquad ( 5 )$$

should approximately have the standard normal distribution N(0,1).

Wilcoxon's rank sum test can be extended to a block of atoms. For example, when all cells have equal sizes, the average of W₂(i)

$${W}_{2} = \frac{1}{n} {\sum }_{i = 1}^{n} {W}_{2} ( i ) \qquad ( 6 )$$

for all atoms in a block can be used as a statistic to make calls.

In one aspect of the invention, methods are provided for comparing a first microarray spot with a second microarray spot. The methods may include steps of providing a first plurality of intensity values (Sᵢ^{A}) for the first microarray spot and a second plurality of intensity values (Sₖ^{B}) for the second microarray spot; calculating a p value using Wilcoxon's rank sum test, where the p value is for a null hypothesis that θ=0 and an alternative hypothesis that θ>0, where θ is a test statistic for intensity difference between the first plurality and the second plurality; and indicating that the first microarray spot is different from the second microarray spot if the p value is greater than a significance level. The test statistic maybe median (Sᵢ^{A})-median( Sₖ^{B}). The significance level can be, for example, 0.01, 0.05 or 0.10. The first microarray spot and second microarray spot may be nucleic acid spots among at least 10, 50, 100, 200, 400, 500, 750, 1,000, 5,000, 10,000, 20,000, 30,000 or more nucleic acid spots on a substrate. The nucleic acid spots are cDNA spots or oligonucleotide spots (either synthesized on the substrate or spotted). In some embodiments, the methods may include combining first plurality and second plurality of intensity values if the p-value is greater than a significance level, such as p>0.5.

In another aspect of the invention, computer software products are provided for comparing a first microarray spot with a second microarray spot. The products comprise computer program code for inputing a first plurality of intensity values ( Sᵢ^{A}) for the first microarray spot and a second plurality of intensity values (Sₖ^{B}) for the second microarray spot; computer program code for calculating a p value using Wilcoxon's rank sum test, where the p value is for a null hypothesis that θ=0 and an alternative hypothesis that the θ>0, where the θ is a test statistic for intensity difference between the first plurality and the second plurality; computer program code for indicating that the first microarray spot is different from the second microarray spot if the p value is greater than a significance level; and a computer readable media for storing the computer program codes. The testing statistic is median (Sᵢ^{A})-median( Sₖ^{B}). The significance level may be, for example, 0.01, 0.05 or 0.10. In preferred embodiments, the computer software products may include computer program code for accepting user's input or selection of the significance level. The computer software products are particularly useful for analyzing spotted nucleic acid arrays such as those having at least 100, preferably at least 1000 nucleic acid spots on a substrate. The nucleic acid spots may be cDNA spots or oligonucleotide spots. The oligonucleotide spots may be spotted or synthesized on the substrate. The computer software products may also include computer program code for combining first plurality and second plurality of intensity values if the p-value is greater than a significance level.

In yet another aspect, systems for comparing two microarray spots are provided. The systems may include a processor; and a memory being coupled to the processor, the memory storing a plurality machine instructions that cause the processor to perform a plurality of logical steps when implemented by the processor, the logical steps including: inputing a first plurality of intensity values (Sᵢ^{A}) for the first microarray spot and a second plurality of intensity values (Sₖ^{B}) for the second microarray spot; calculating a p value using Wilcoxon's rank sum test, where the p value is for a null hypothesis that θ=0 and an alternative hypothesis that the θ>0, where the θ is a test statistic for intensity difference between the first plurality and the second plurality; and indicating that the first microarray spot is different from the second microarray spot if the p value is greater than a significance level. The testing statistic may be median (Sᵢ^{A})-median(Sₖ^{B}). The significance level may be 0.05. In some preferred embodiments, the steps further include accepting user's input or selection of the significance level.

The systems are particularly useful for analyzing spotted nucleic acid arrays such as those having at least 10, 50, 100, 200, 400, 500, 750, 1,000, 5,000, 10,000, 20,000, 30,000 or more nucleic acid spots on a substrate. The nucleic acid spots may be cDNA spots or oligonucleotide spots. The oligonucleotide spots may be spotted or synthesized on the substrate. The computer software products may also include computer program code for combining first plurality and second plurality of intensity values if the p-value is greater than a significance level.

Another use is characterizing experimental repeatability. The 3 spots: 135 nM A, 135 nM B and 135 nM C are replicates. The results of Table 1 show that the spot intensities are not the same and the method characterizes their intensity differences.

Another use is the ability to know whether observed intensity differences are due to mRNA differences or merely due to experimental variability. For the example data (Table 1), p-values more than approximately 0.0363 are probably due merely to experimental varibility and should not be assigned to further interpretation.

Yet another use is the ability to know whether observed signal intensity is significantly larger than a background intensity. In some embodiments, if a signal intensity (derived from a probe against a transcript of a gene) is detected as significantly higher than a background, the expression of the gene is detected. In this use, the set of pixels from the spot would be compared with the set of pixels representing the background intensity using the Wilcoxon rank sum test. The methods of the invention are not limited to any particular method of selecting the background pixels.

In some embodiments, the methods, software and systems are used to evaluate other intensity analysis (such as parametric analysis) algorithm. The parametric results should be in agreement with the nonparametric results. That is, for two spots, the spot with the larger mean rank (nonparametric result) should normally have the larger intensity.

Methods, computer software products and systems are also provided for analyzing determining whether a transcript is present in a biological sample using nucleic acid probe arrays that have probes designed to be complementary to the transcript (perfect match probe, PM) and probes that are designed to contain mismatch against the transcript (mismatch probe, MM). The methods include providing a plurality of perfect match pixel intensity values (PMᵢⱼ) and mismatch pixel intensity values (MMᵢₖ) for the transcript, where the PMᵢⱼ is the pixel intensity value for perfect match probe i and pixel j and MMᵢₖ is the pixel intensity value for mismatch probe i and pixel k; calculating a p-value using one-sided Wilcoxon's rank sum test, wherein the p-value is for a null hypothesis that (median(PMᵢⱼ)-median(MMᵢₖ))=a threshold value and an alternative hypothesis that (median(PMᵢⱼ)-median(MMᵢₖ))>the threshold value; and indicating whether the transcript is present based upon the resulting p-value. In some embodiments, the threshold value is zero. In some other preferred embodiments, the threshold value is calculated using:

τ=c\sqrt{median(PMᵢ)} or τ=c₁\sqrt{mean(PMᵢ)}

where c is a constant.

The presence, marginal present or absence (detected, marginally detected or undetected) of a transcript may be called based upon the p-value and significance levels. Significance levels, α₁ and α₂ may be set such that: 0<α₁<α₂<0.5. Note that for the one-sided test, if null hypothesis is true, then the most likely observed p-value is 0.5, which is equivalent to 1 for the two-sided test. Let p be the p-value of one sided rank sum test. In preferred embodiments, if p<α₁, a “detected” call can be made (i.e., the expression of the target gene is detected in the sample). If α₁≦p<α₂, a marginally detected call may be made. If p≧α₂, “undetected call” may be made. The proper choice of significance levels and the thresholds can reduce false calls.

Some preferred embodiments of the computer software product for determining whether a transcript is present in a biological sample include computer program code for inputting a plurality of perfect match pixel intensity values (PMᵢⱼ) and mismatch pixel intensity values (MMᵢₖ) for the transcript, wherein the PMᵢⱼ is the pixel intensity value for perfect match probe i and pixel j and MMᵢₖ is the pixel intensity value for mismatch probe i and pixel k; computer software code for calculating a p-value using one-sided Wilcoxon's rank sum test, wherein the p-value is for a null hypothesis that (median(PMᵢⱼ)-median(MMᵢₖ))=a threshold value and an alternative hypothesis that (median(PMᵢⱼ)-median(MMᵢₖ))>threshold value; computer software code for indicating whether the transcript is present based upon said p-value; and a computer readable media for storing the codes.

In some embodiments, the threshold value is zero. In some other preferred embodiments, the threshold value is calculated using:

τ=c\sqrt{median(PMᵢ)} or τ=c₁\sqrt{mean(PMᵢ)}

where c is a constant.

The computer software product may also include code for indicating the presence, marginal presence or absence of the transcript based up the p-value and significance level. Appropriate significance level may be pre-set or inputted by a user.

The systems for comparing nucleic acid probes may include a processor; and a memory being coupled to the processor, the memory storing a plurality of machine instructions that cause the processor to perform a plurality of logical steps when implemented by the processor, the logical steps including: providing a plurality of perfect match pixel intensity values (PMᵢⱼ) and mismatch pixel intensity values (MMᵢₖ) for the transcript, where PMᵢⱼ is the pixel intensity value for perfect match probe i and pixel j and MMᵢₖ is the pixel intensity value for mismatch probe i and pixel k;calculating a p-value using one-sided Wilcoxon's rank sum test, wherein the p-value is for a null hypothesis that (median(PMᵢⱼ)-median(MMᵢₖ))=a threshold value and an alternative hypothesis that said (median(PMᵢⱼ)-median(MMᵢₖ))>said threshold value; and indicating whether said transcript is present based upon said p-value.

In some embodiments, the threshold value is zero. In some other preferred embodiments, the threshold value is calculated using:

τ=c\sqrt{median(PMₜ)} or τ=c₁\sqrt{mean(PMᵢ)}

where c is a constant.

The presence, marginal present or absence (detected, marginally detected or undetected) of a transcript may be called based upon the p-value and significance levels. Significance levels, α₁ and α₂ may be set such that: 0<α₁<α₂<0.5. Note that for the one-sided test, if null hypothesis is true, the most likely observed p-value is 0.5, which is equivalent to 1 for the two-sided test. Let p be the p-value of one sided rank sum test. In preferred embodiments, if p<α₁, a “detected” call can be made (i.e., the expression of the target gene is detected in the sample). If α₁≦p<α₂, a marginally detected call may be made. If p≧α_{2,} “undetected call” may be made. The proper choice of significance levels and the thresholds can reduce false calls.

IV. Example

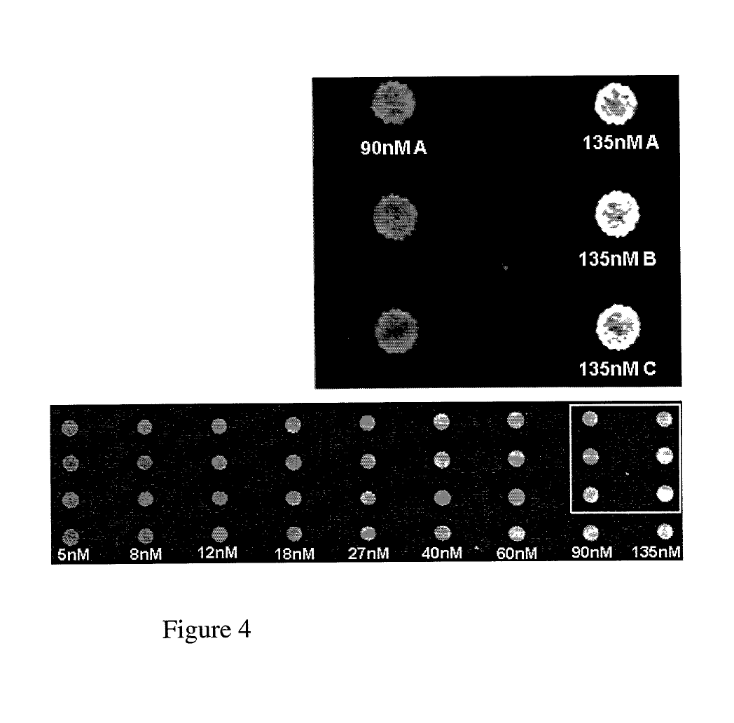

The methods of using Wilcoxon's rank sum test will be illustrated using the following example. FIG. 4 shows an image of microarray spots. The highlighted portion of the data is expanded in size and in gray scale to show details. The image annotations were added for clarification and are not part of the original data analyzed.

The pixel intensities for the two sets are organized as follows. Assign all the intensities from one of the spots, for example: 135 nM A to set S^{A}. Assign all intensities from the other spot, for example 135 nM B to S^{B}. Let n be the size of S^{A} (in this case spot 135 NM A has 174 pixels). Let m be the size of S^{A} (in this example spot 135 nM B has 198 pixels). Let the i-th pixel intensity in S^{A} be Sᵢ^{A} (i=1, . . . n). Let the k-th pixel intensity in S^{B} be Sₖ^{B} (k=1, . . . m).

The combined pixel intensity data, S^{A} and S^{B} can be sorted and ranked with integers 1,2, . . . p, where p=m+n (in this case 174+198=372). If there are ties (in this case there were 5), the average of the integer ranks for all elements in a tie group may be used. Let the rank of Sᵢ^{A} be Rᵢ^{A} and the rank of Sₖ^{B} be Rₖ^{B}. The rank sum may be calculated as:

$$W = {\sum }_{j = 1}^{n} {R}_{i}^{A}$$

In this example, W was 30285 for 135 nM A. The exact p-value of the observed W for the null hypothesis (the probability that the two spots are actually the same intensity) can be calculated (p=0.0363 for this example). In the specific example, the probability that the two spots have the same intensity was 3.63%; therefore the probability that they are of different intensities is 100% minus 3.63% or 96.73%.

TABLE 1
Example Results, Comparing Spot Intensity Data

| Comparison | | Probability Spots have Different | Mean |
|---|---|---|---|
| Spots | p-value | Intensities | Ranks |
| 135nM A | 0.0363 | 97.37% | 174.1 |
| 135nM B | | | 197.4 |
| 135nM A | 0.6417 | 35.83% | 183.7 |
| 135nM C | | | 188.9 |
| 135nM A | <0.0001 | >99.99% | 229.3 |
| 90nM A | | | 103.2 |

The results shown in Table 1 confirm what is visible from the data in FIG. 4. That is, of the 3 comparisons, Spot 135 nM A is most different in intensity from spot 90 nM A. Furthermore, careful inspection of the data in FIG. 4 shows that indeed spot 135 nM A is more similar in intensity to spot 135 nM C than to spot 135nM B as Table 1 shows.

The example data shown in FIG. 2 and Table 1 suggest several uses of this method.

The method correctly agrees with the obvious observation that spot 135 nM A is very different in intensity from spot 90 nM A. Furthermore, the mean ranks also agree 135 nM A mean rank is larger than 90 nM A mean rank) with the observation that 135 nM A is the brighter spot.

Another use is characterizing experimental repeatability. The 3 spots: 135 nM A, 135 nM B and 135 nM C are replicates. The results of Table 1 show that the spot intensities are not the same and the method characterizes their intensity differences.

Another use is the ability to know whether observed intensity differences are due to mRNA differences or merely due to experimental variability. For the example data (Table 1), p-values more than approximately 0.0363 are probably due merely to experimental variability and should not be assigned to further interpretation.

Another use is combining replicate spots into one distribution for intensity comparisons. For example, spots 135 nM A, 135 nM B and 135 nM C intensity data could be combined into one data set, S₁ and then compared to another data set S₂ using this method. Combining replicate spots may allow more information to be extracted from the intensity data.

Another use is evaluating an intensity determination (parametric) algorithm. The parametric results should be in agreement with the nonparametric results. That is, for two spots, the spot with the larger mean rank (nonparametric result) should also have the larger intensity.

After a comparison is made the data is preferably analyzed for biologically relevant information. For example, further data analysis would be useful in gene expression monitoring, genotyping and other polymorphism analysis, diagnostics, etc.

### Conclusion

The present inventions provide methods and computer software products for analyzing gene expression profiles. It is to be understood that the above description is intended to be illustrative and not restrictive. Many variations of the invention will be apparent to those of skill in the art upon reviewing the above description. By way of example, the invention has been described primarily with reference to the use of a high density oligonucleotide array, but it will be readily recognized by those of skill in the art that other nucleic acid arrays, other methods of measuring transcript levels and gene expression monitoring at the protein level could be used. The scope of the invention should, therefore, be determined not with reference to the above description, but should instead be determined with reference to the appended claims, along with the full scope of equivalents to which such claims are entitled.

All cited references, including patent and non-patent literature, are incorporated herewith by reference in their entireties for all purposes.

What is claimed is:

1. A method for comparing a first microarray spot with a second microarray spot comprising:
providing a first plurality of intensity values (Sᵢ^{A}) for said first microarray spot and a second plurality of intensity values (Sₖ^{B}) for said second microarray spot;
calculating a p value using Wilcoxon's rank sum test, wherein said p value is for a null hypothesis that θ=0 and an alternative hypothesis that said θ>0, wherein said θ is a test statistic for intensity difference between said first plurality and said second plurality; and
indicating said first microarray spot is different from said second microarray spot if said p value is greater than a significance level.

2. The method of claim 1 wherein said testing statistic is median (Sᵢ^{A})-median( Sₖ^{B}).

3. The method of claim 2 wherein said significance level is 0.05.

4. The method of claim 1 wherein said first microarray spot and second microarray spot are nucleic acid spots.

5. The method of claim 4 wherein said nucleic acid spots are among at least 100 nucleic acid spots on a substrate.

6. The method of claim 5 wherein said nucleic acid spots are among at least 1000 spots on said substrate.

7. The method of claim 6 wherein said nucleic acid spots are cDNA spots.

8. The method of claim 7 wherein said nucleic acid spots are oligonucleotide spots.

9. The method of claim 1 further comprising step of combining first plurality and second plurality of intensity values if said p-value is greater than a significance level.

10. A computer software product for comparing a first microarray spot with a second microarray spot comprising:
computer program code for inputing a first plurality of intensity values (Sᵢ^{A}) for said first microarray spot and a second plurality of intensity values (Sₖ^{B}) for said second microarray spot;
computer program code for calculating a p value using Wilcoxon's rank sum test, wherein said p value is for a null hypothesis that θ=0 and an alternative hypothesis that said θ>0, wherein said θ is a test statistic for intensity difference between said first plurality and said second plurality; and
computer program code for indicating said first microarray spot is different from said second microarray spot if said p value is greater than a significance level; and
a computer readable media for storing said computer program codes.

11. The computer program product of claim 10 wherein said testing statistic is median (Sᵢ^{A})-median(Sₖ^{B}).

12. The computer program of claim 11 wherein said significance level is 0.05.

13. The computer software product of claim 11 further comprising computer program code for accepting user's input or selection of said significance level.

14. The computer software product of claim 11 wherein said first microarray spot and second microarray spot are nucleic acid spots.

15. The computer software product of claim 14 wherein said nucleic acid spots are among at least 100 nucleic acid spots on a substrate.

16. The computer software product of claim 15 wherein said nucleic acid spots are among at least 1000 spots on said substrate.

17. The computer software product of claim 16 wherein said nucleic acid spots are cDNA spots.

18. The computer software product of claim 16 wherein said nucleic acid spots are oligonucleotide spots.

19. The computer software product of claim 10 further computer program code for combining first plurality and second plurality of intensity values if said p-value is greater than a significance level.

20. The computer software product of claim 19 wherein said significance level is 0.5.

21. A system for comparing nucleic acid probes, comprising:
a processor; and
a memory being coupled to the processor, the memory storing a plurality machine instructions that cause the processor to perform a plurality of logical steps when implemented by the processor, said logical steps including:
inputing a first plurality of intensity values (Sᵢ^{A})for said first microarray spot and a second plurality of intensity values (Sₖ^{B}) for said second microarray spot;
calculating a p value using Wilcoxon's rank sum test, wherein said p value is for a null hypothesis that θ=0 and an alternative hypothesis that said θ>0,
wherein said θ is a test statistic for intensity difference between said first plurality and said second plurality; and
indicating said first microarray spot is different from said second microarray spot if said p value is greater than a significance level.

22. The system of claim 21 wherein said testing statistic is median (Sᵢ^{A}) -median( Sₖ^{B}).

23. The system of claim 22 wherein said significance level is 0.05.

24. The system of claim 22 wherein said steps further comprise accepting user's input or selection of said significance level.

25. The system of claim 21 wherein said first microarray spot and second microarray spot are nucleic acid spots.

26. The system of claim 25 wherein said nucleic acid spots are among at least 100 nucleic acid spots on a substrate.

27. The system of claim 26 wherein said nucleic acid spots are among at least 1000 spots on said substrate.

28. The system of claim 27 wherein said nucleic acid spots are cDNA spots.

29. The system of claim 27 wherein said nucleic acid spots are oligonucleotide spots.

30. The system of claim 21 wherein said steps further comprise combining first plurality and second plurality of intensity values if said p-value is greater than a significance level.

31. The system of claim 30 wherein said significance level is 0.5.

32. A method for determining whether a transcript is present in a biological sample comprising:
providing a plurality of perfect match pixel intensity values (PMᵢⱼ) and mismatch pixel intensity values (MMᵢₖ for the transcript, wherein said PMᵢⱼ is the pixel intensity value for perfect match probe i and pixel j and MMᵢₖ is the pixel intensity value for mismatch probe i and pixel k;
calculating a p-value using one-sided Wilcoxon's rank sum test, wherein the p-value i s for a null hypothesis that (median (PMᵢⱼ)-median(MMᵢₖ))=a threshold value and an alternative hypothesis that said (median(PMᵢⱼ)-median(MMᵢₖ))>said threshold value; and
indicating whether said transcript is present based upon said p-value.

33. The method of claim 32 wherein said threshold value is zero.

34. The method of claim 32 wherein said threshold value is calculated using:
τ=c\sqrt{median(PMᵢ)}
wherein said c is a constant.

35. The method of claim 32 wherein threshold value is calculated using:
τ=c₁\sqrt{mean(PMᵢ)}
wherein said c is a constant.

36. The method of claim 32 wherein said step of indicating comprises indicating said transcript is present if said p is smaller than a first significance level (α₁).

37. The method of claim 32 wherein said step of indicating further comprises indicating said transcript is absent if said p is greater than or equal to a second significance level (α₂).

38. The method of claim 37 wherein said step of indicating further comprises indicating said transcript is marginally detected if α₁≦p<α₂.

39. A computer software product for determining whether a transcript is present in a biological sample comprising:
computer program code for inputting a plurality of perfect match pixel intensity values (PMᵢⱼ) and mismatch pixel intensity values (MMᵢₖ) for said transcript, wherein said PMᵢⱼ is the pixel intensity value for perfect match probe i and pixel j and MMᵢₖ is the pixel intensity value for mismatch probe i and pixel k;
computer software code for calculating a p-value using one-sided Wilcoxon's rank sum test, wherein the p-value is for a null hypothesis that (median(PMᵢⱼ)-median(MMᵢₖ))=a threshold value and an alternative hypothesis that said (median(PMᵢⱼ)-median(MMᵢₖ))>said threshold value;
computer software code for indicating whether said transcript is present based upon said p-value; and
a computer readable media for storing said code.

40. The computer software product of claim 32 wherein said threshold value is zero.

41. The computer software product of claim 32 wherein said threshold value is calculated using:
τ=c\sqrt{median(PMᵢ)}
wherein said c is a constant.

42. The computer software product of claim 32 wherein threshold value is calculated using:
τ=c₁\sqrt{mean(PMᵢ)}
wherein said c is a constant.

43. The computer software product of claim 32 wherein said computer program code for indicating comprises computer software code for indicating that said transcript is present if said p is smaller than a first significance level (α₁).

44. The computer software product of claim 32 wherein said computer program code for indicating further comprises computer software code for indicating said transcript is absent if said p is greater than or equal to a second significance level (α₂).

45. The computer software product of claim 37 wherein said computer program code for indicating further comprises computer software code for indicating that said transcript is marginally detected if α₁≦p<α₂.

46. A system for comparing nucleic acid probes, comprising:
a processor; and
a memory being coupled to the processor, the memory storing a plurality machine instructions that cause the processor to perform a plurality of logical steps when implemented by the processor, said logical steps including:
providing a plurality of perfect match pixel intensity values (PMᵢⱼ) and mismatch pixel intensity values (MMᵢₖ) for the transcript, wherein said PMᵢⱼ is the pixel intensity value for perfect match probe i and pixel j and MMᵢₖ is the pixel intensity value for mismatch probe i and pixel k;
calculating a p-value using one-sided Wilcoxon's rank sum test, wherein the p-value is for a null hypothesis that (median(PMᵢⱼ)-median(MMᵢₖ))=a threshold value and an alternative hypothesis that said (median(PMᵢⱼ)-median(MMᵢₖ))>said threshold value; and
indicating whether said transcript is present based upon said p-value.

47. The system of claim 46 wherein said threshold value is zero.

48. The system of claim 47 wherein said threshold value is calculated using:
τ=c\sqrt{median(PMᵢ)}
wherein said c is a constant.

49. The system of claim 47 wherein threshold value is calculated using:
τ=c₁\sqrt{mean(PMᵢ)}
wherein said c is a constant.

50. The system of claim 46 wherein said step of indicating comprises indicating said transcript is present if said p is smaller than a first significance level (α₁).

51. The system of claim 50 wherein said step of indicating further comprises indicating said transcript is absent if said p is greater than or equal to a second significance level (α₂).

52. The system of claim 51 wherein said first significance level (α₁) is smaller than said (α₂) and said step of indicating further comprises indicating said transcript is marginally detected if (α₁≦p<α₂.
